(19) United States
(12) Patent Application Publication
(10) Pub. No.: US 2002/0049479 A1
(43) Pub. Date: Apr. 25, 2002

(54) Method and apparatus for creating afferents to prevent obstructive sleep apnea

(76) Inventor: Walter C. Pitts, La Quinta, CA (US)

Correspondence Address: CHRISTIE, PARKER & HALE, LLP, 350 WEST COLORADO BOULEVARD, SUITE 500, PASADENA, CA, 91105, US

(21) Appl. No.: 09/954,315

(22) Filed: Sep. 17, 2001

Related U.S. Application Data
(60) Provisional application No. 60/241,932, filed on Oct. 20, 2000.

(51) Int. Cl. A61N 1/36

(52) U.S. Cl. 607/42

(57) ABSTRACT

A method and device for creating an afferent stimulus for preventing obstructive sleep apnea are disclosed. The device includes at least one implantable electrode and a stimulator, of which at least one electrode is implanted in the genioglossus muscle of a patient having obstructive sleep apnea. The electrode is capable of conducting selected electrical stimulation generated by the stimulator, and the system is capable of delivering the selected electrical stimulation during a selected time of day. The electrical stimulation is selected to maintain sufficient muscle tone of the genioglossus muscle to prevent it from obstructing the airway during sleep, preferably at a stimulus intensity low enough to avoid awakening the patient during sleep.

### CROSS-REFERENCE TO RELATED APPLICATION(S)

This application claims priority to Provisional Application No. 60/241,932, filed Oct. 20, 2000.

### BACKGROUND OF THE INVENTION

Obstructive sleep apnea (OSA) is a disorder resulting from an affected individual's upper airway being obstructed or partially obstructed during sleep causing arousals from sleep. In obstructive apnea, the airflow stops, but the effort by the diaphragm continues. The individual stops breathing for many seconds during sleep and may awake repeatedly with a loud snore or gasp for breath.

OSA results from excessive relaxation of the upper airway muscles during sleep, coupled with an unknown dysfunction of respiratory neurons. The air on its way to the lungs passes through the oropharynx and hypopharynx, and in OSA, not only are the pharyngeal muscles affected, but the base of the tongue collapses posteriorly against the lower oropharynx and the upper hypopharynx. Ordinarily, reflex activity works against this collapse during wakefulness to maintain patency of the airway. However, the process is further complicated by negative pressure in the airway during sleep. Thus, OSA occurs if the tissues in the airway periodically collapse and the airway becomes occluded in varying dimensions to result in snoring, hypopneas, and apneas.

OSA is the most common form of apnea. Patients with OSA stop breathing many times during sleep, measured as cessation of breathing for longer than ten seconds by a nighttime polysomnogram (NPSG) in a sleep disorder laboratory. Patients make gasping or snorting sounds which may or may not completely awaken them, but more importantly, creates an EEG arousal which contributes to excessive daytime sleepiness.

There is also a family history of apnea, which may be due to inherited physical craniofacial characteristics, such as retrognathia, which can cause breathing abnormalities, such as snoring, hypopneas, and apneas. Obesity has been associated with sleep apnea because fatty cells infiltrate the throat tissue, which may cause a narrowing of the airways and increase the risk for sleep apnea. While OSA occurs more frequently in overweight men, both genders are affected and even men and women with body mass indexes (BMI) in the range between 25 and 30 suffer from OSA. Contributing factors may include use of alcohol or sedatives before sleep, anatomically narrowed airways, and massively enlarged tonsils and adenoids. Hypertension or pulmonary hypertension with enlarged right ventricle may be present. Persistent low levels of oxygen (hypoxia) cause daytime symptoms such as hypersomnolence, headaches, intellectual deterioration, and cardiac arrhythmias. If the condition is severe enough patients are at risk for stroke and heart attack.

Historically, treatment of obstructive sleep apnea syndrome initially consisted of avoidance of sedatives or alcohol consumption, and weight loss. The objective of treatment is to keep the airway open to prevent apneic episodes during sleep. Weight management (or intentional weight loss) and the avoidance of alcohol and sedatives at bedtime may achieve the desired results in some individuals. If these measures are unsuccessful in stopping sleep apnea, continuous positive airway pressure (CPAP), involving the use of a specially designed mask worn over the nose at night, with air pressure applied through tubing into the airway to keep the airway from collapsing, may be prescribed. Alternatively, mechanical devices such as intra-oral airway dental prostheses, may be used. They are inserted into the mouth at night to keep the jaw forward. Oxygen therapy in select cases may achieve the desired results. Finally, surgery (e.g., uvulopalatopharyngoplasty (UPPP), laser assisted uvuloplasty (LAUP), and somnoplasty) to remove soft palate tissue, or tracheostomy to create an opening in the trachea to bypass the obstructed airway during sleep has been performed on some patients with refractory OSA.

However, behavioral therapies (e.g., weight loss, eliminating central depressant use) are limited by patient compliance, and perhaps anatomical constraints. Physical interventions such as CPAP masks and dental prostheses are also limited by patient compliance, as they may be uncomfortable and inconvenient. For example, CPAP consists of an airway mask or nasal pillows attached to a machine which delivers continuous air to the pharyngeal airway space to reverse negative airway pressure at the base of the tongue in the hypopharynx. The mask must be worn all night, disconnected when going to the bathroom, then reconnected. Patients complain of discomfort wearing the mask during sleep, claustrophobia, and marks on their face in the morning because of the tightness of the mask. Compliance with this treatment is estimated at between 40-60%.

Intra-oral airway dental devices are oral splints which advance the lower jaw forward into a protrusive position to prevent the tongue from falling against the posterior airway in the lower oropharynx and upper hypopharynx. Some patients complain of discomfort wearing an oral prosthesis at night. Compliance with this treatment modality is also between about 40-60%, and it is less effective than CPAP at resolving apnea.

Surgical interventions, such as UPPP, LAUP, and somnoplasty, have the lowest success rate at resolving obstructive sleep apnea (between 10-20%), because the site of obstruction is generally lower in the pharyngeal region than the site of the surgery, which is done higher in the airway at the soft palate. Additionally, post-surgical recovery is generally quite painful and protracted.

Electrical stimulation methods and devices have been developed in an effort to eliminate the obstruction of the airway by contraction of the upper airway musculature. Such devices generally sense an apneic event by monitoring breathing, and when the absence thereof is detected, apply stimulation to nerves or muscles of the upper airway to move them away from the center of the airway. Such methods and devices suffer from disadvantages such as awakening or arousal of the patient (by the stimulation and muscle contraction, or because the devices themselves are uncomfortable), and they do not prevent the apneic event. Therefore, they do not resolve problems of fragmented sleep or patient compliance.

Current therapies directed at treating OSA suffer from quality of life limitations for the patients using them. A need clearly exists for a more elegant and sophisticated treatment. Preferably, such an alternative does not interrupt the sleep of the patient, and thus eliminates the daytime effects of inadequate sleep, as well as the physiological effects of airway occlusion.

### SUMMARY OF THE INVENTION

The instant invention provides a system and method of preventing obstructive sleep apnea that is both comfortable for the patient, does not require bulky apparatus, and allows the patient to get uninterrupted sleep. Thus, patient compliance is high, and all symptoms of obstructive sleep apnea are mitigated.

The invention includes a method of preventing obstructive sleep apnea events by providing a system which comprises at least one implantable electrode and a stimulator, and implanting the electrode(s) intraorally, so that the genioglossus of a patient is preferably stimulated in the posterior one-third of the tongue, posterior to the sulcus terminalis. The electrode is capable of conducting selected electrical stimulation generated by the stimulator and delivering the selected electrical stimulation during a selected time of day. The electrical stimulation is selected to maintain sufficient muscle tone of the muscles of the upper airway so that the airway does not become obstructed. In the method, the muscle maintaining tone is the genioglossus muscle and/or muscles of the upper airway (pharynx). In some embodiments, the electrode is placed near enough to the glossopharyngeal nerve that stimulation effects glossopharyngeal branches (efferents and/or afferents), thus inducing muscle tone in the airway muscle fiber served by the stimulated branch of the glossopharyngeal. The preferred implantation site is intraorally so that the posterior one third of the genioglossus muscle is stimulated by the electrode and/or device.

In preferred embodiments, the system further comprises a controller, which preferably turns the stimulator on or off, and/or sets or modifies stimulus parameters. Preferably, the system is activated and de-activated at pre-determined times, such as when the patient goes to bed, although it can be left on at all times, since depolarization of the affected muscles is minimal. The stimulus is provided at an intensity high enough to produce sufficient muscle tone that tissues of the airway do not prolapse into the airway, and preferably the muscle tone approximates normal waking muscle tone. The stimulus is also preferably low enough in intensity that the patient can sleep through the stimulus and attendant muscle depolarization, either because of habituation to the stimulus or because it cannot be perceived much, if at all.

### DESCRIPTION OF THE DRAWINGS

FIG. 1 indicates positions at the base of the tongue (genioglossus muscle) where electrodes or microstimulator devices can be implanted to deliver current for muscle tone maintenance.

FIG. 2 is a lateral view of the tongue and local structures, indicating a preferred electrode or microstimulator implantation site.

FIG. 3 is a posterior view of the base of the tongue indicating one preferred stimulation site.

### DETAILED DESCRIPTION OF THE INVENTION

The methods and system of the invention provide a surgically implanted electrode or microdevice capable of stimulating the genioglossus muscle (tongue) in the back or lower one-third to one-half of the muscle such that muscle tone is maintained throughout the night, preventing the occurrence of any obstructive sleep apnea events and promoting uninterrupted sleep. Other muscles in the airway may also be implanted and stimulated, or may be affected by glossopharyngeal stimulation according to the invention. Unlike other electrical stimulation methods, the inventive methods are proactive rather than responsive to obstructive events that cause a cessation in breathing, so that rather than a treatment modality, the invention provides a preventative therapy. The implantation is surgical, so patient compliance is high, and because the electrical stimulation used is generally at lower intensities than prior art methods, the patient is more comfortable and less likely to awaken from sleep due to the stimulation.

The system includes at least one implantable electrode (which may be suitable for chronic implantation), a pulse generator or stimulator, and a control to turn the stimulator on or off and to modulate the frequency, amplitude, intensity and the like delivered by the stimulator to the electrode. Very small devices (“microdevices,” “MicroElectroMechanical Systems (also known as MEMS, micromachines, microactuators, or microsensors),” or “microbiosensors”), having capabilities similar to cardiac pacemakers and brain stimulators, are now available that combine stimulator and electrode functions. MicroElectroMechanical Systems are physically quite small, and are therefore suitable for implantation in the genioglossus muscle. An array of small stimulators (or sensors) can also be used for redundancy. MEMS are useful as actuators or stimulators because the stimuli they deliver can be very precise. An example is described in “Monolithic Microfabricated Valves and Pumps by Multilayer Soft Lithography” (Unger et al., Science (2000) Apr. 7; 288:113-116).

In a preferred embodiment, a medical device is implanted to deliver mild electrical stimulation to produce muscle tone without full contraction. It involves implanting a thin, wire (an electrode, or a “lead”), or the entire device, if it is small enough, in one or more selected locations in the lower one third of the genioglossus muscle. Preferably, the device is placed in or near the posterior one-third of the tongue, submentally below the tongue to maintain muscle tone of the hyoid muscles, or even in the clavicular region. All locations necessitate electrode leads to deliver the proper amount of stimulation for muscle tonicity.

If the device is not sufficiently small as to be implanted in the genioglossus muscle without physical effects, then the electrode or lead is implanted alone. In such an embodiment, the lead is connected by an extension to a stimulator/pulse generator, which has a battery and suitable electronics. The stimulator is generally implanted nearby, for example, near the collarbone. The stimulation level can be adjusted as needed to get the best possible muscle tone with minimal contraction. The therapy is reversible because the system can be turned off or removed.

Suitable materials for implantable electrodes and/or microdevices are those which are biocompatible with tissues for chronic implantation and will not promote excessive immune reaction or scar tissue formation. “Electrodes” as used generally herein can include both separate implantable electrodes, and current delivery contact point or points on an implanted microdevice. Suitable materials for electrodes include for example iridium, platinum, titanium, rhodium, gold, carbon, and oxides of these elements (e.g., iridium/iridium oxide). Examples of implantable electrodes and methods of fabrication are described in U.S. Pat. No. 5,524,338, herein incorporated by reference. Alternatively, technology as is well known for cardiac pacemakers can be adapted for use in the invention, for example, an implanted stimulator with battery power and control electronics (at a site remote from the genioglossus muscle if the implant is larger than can be accommodated easily within the genioglossus muscle) and leads bearing electrodes leading to the implantation site in the genioglossus muscle.

Ideal characteristics of the electrode or electrode array (e.g., size, shape, number of contact sites) varies depending on the location and tissue type and characteristics (e.g., nerve or muscle, and number of motor units affected) where it is to be implanted. However, one or essentially any number of electrodes may be used in an array or microdevice, and many electrode configurations are suitable, such as wire or plate electrodes, deformable insulated (with the insulation removed at desired contact points) or uninsulated wire mesh. Plate or mesh electrodes can be of a size suitable to stimulate the desired area, or if insulated, can have uninsulated contact regions of any desired area.

The electrodes, if used in an array, can pass current for a given stimulus protocol wired in parallel to deliver the applied current protocol simultaneously from one source, or each active electrode can be independently connected to a stimulating device, allowing each electrode to deliver the same or a different protocol on any time interval or with any phase shift desired. Stimulus protocols can be any impulse or stimulus train that prevents the tongue from relaxing into the airway. Preferably, stimuli are delivered at the lowest possible intensity and frequency, with the preferred goal being maintenance of sufficient tone in the genioglossus muscle to prevent its prolapse into the airway, and prevention of sleep disruption. More preferably, the stimuli are sufficient to maintain tone, but insufficient to cause generalized muscle contraction.

The electrode or electrodes are placed to affect the minimum number of individual muscle fibers necessary to maintain overall genioglossus muscle position in the open airway. Syncytial or cell to cell current transfer effects are considered when deciding on the number of electrodes to implant, depending on the stimulus intensity desired. Electrodes can be placed near or in contact with branches of the glossopharyngeal nerve (efferents or afferents), so that stimulation affects nerve conduction leading to one or more motor units not in contact with the electrode. As used herein, “motor units,” “muscle” and “muscle fibers” are intended to mean muscle cells. Alternatively, electrodes are placed in contact with or in the area of muscle to be affected. Stimulation is preferably set to depolarize muscle fibers only enough to maintain tone, although it is understood that some or all motor units in an affected area may depolarize more completely and contract, without deviating from the scope of the invention. Preferably, stimulation and muscle depolarization, leading to maintenance of tone and including any muscle fiber contraction, is not sufficiently severe to interrupt a patient's sleep. This effect may be achieved through patient habituation to the stimulus, or because it is of sufficiently low intensity as to be minimally or imperceptible to the patient.

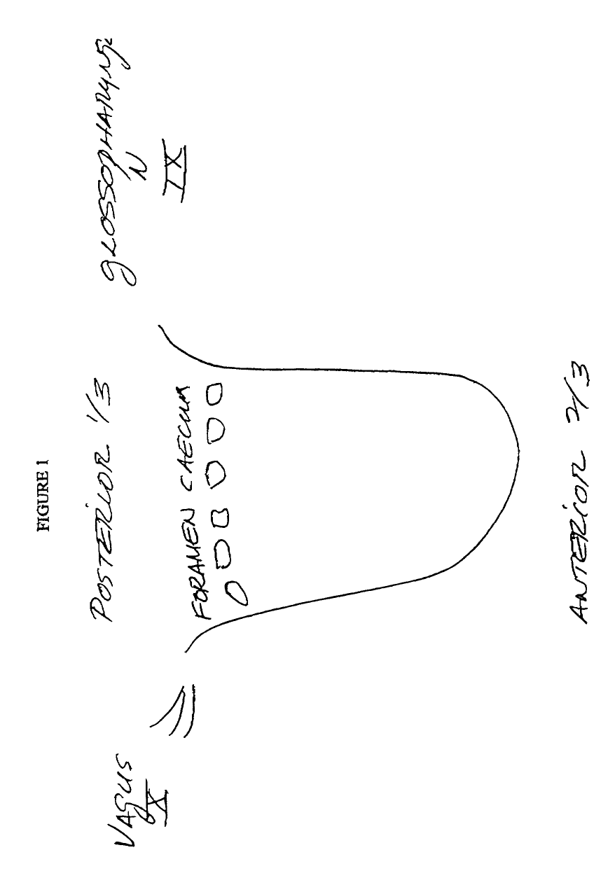

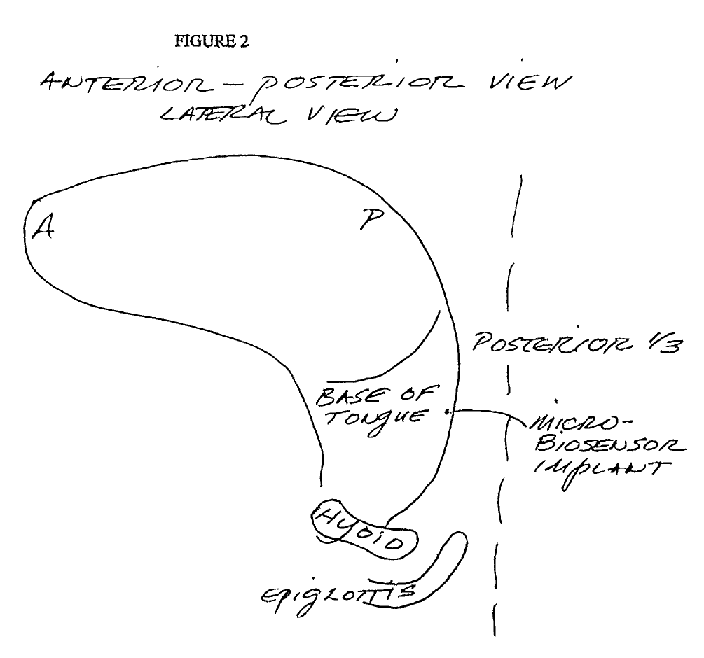

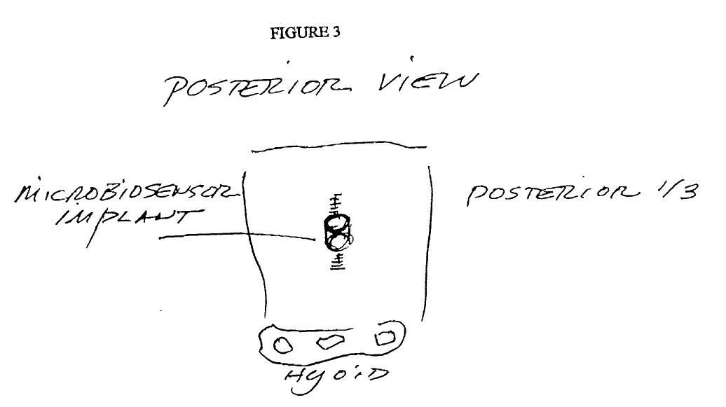

The electrode or microdevices having current delivery and control capability are implanted, preferably chronically, in or near the posterior one third to one half of the genioglossus muscle, around the sulcus terminalis, e.g., either superficially or deep within the muscle (FIGS. 1-3), or at locations intersecting with fibers or branches of the glossopharyngeal nerve innervating the genioglossus muscle. If the electrode is not placed within the musculature of the genioglossus muscle, it is preferably placed in close enough proximity to stimulate fibers of the genioglossus muscle or nerve fibers innervating it.

In a preferred embodiment, the surgical incisions are placed distal to the sulcus terminalis of the tongue. These incisions are therefore located at the base of the tongue. The pre-programmed microdevice is then placed at the incisal location. The patient therefore does not have any responsibility in compliance. The microdevice will maintain the muscle tone of the tongue and pharyngeal muscles as when awake.

As described above, the electrode may be separate from or part of the stimulating device, and simply refers to the portal of current delivery to the nerve or muscle of interest. The MEMS or other microdevice has the capability of delivering stimuli upon the demand of a control unit, or being programmable to deliver the type and duration of stimuli required at desired times. Sensor functions are optional in the microdevices, but may be incorporated to monitor physiological functions such as breathing, muscle tone or contraction, blood gases or pH, and the like. If a separate control function is used, it can be any known in the art (e.g., magnetic, electromagnetic or radiofrequency communication with the implanted device) to turn the stimulating function on or off, to program different stimulation protocols, or to vary the stimulation parameters, such as amplitude and frequency of delivered stimuli. If the device is programmable, stimulus parameters can be determined empirically on a patient-by-patient basis for optimal genioglossus muscle tone. If the device is not programmable, it can be set at parameters determined to be effective at maintaining tone in most patients.

Protocols will generally be uncomplicated, for example, repetitive stimulation will preferably be just sufficient to maintain muscle tonicity. For example, repetitive stimulation when the device is turned on will preferably be between about 0.001 Hz to about 100 Hz. Alternatively, stimulus trains with breaks may be employed, or step functions, decaying biphasic waveforms, etc.

The device(s) is turned on as the patient goes to bed, and delivers low level stimulation to the genioglossus muscle or branches of the ninth cranial (glossopharyngeal) nerve that innervate motor units of the tongue and other pharyngeal muscles. By “low level” stimulation is meant either subthreshold or threshold stimulation sufficient to induce a muscle tone characteristic of an awake person or a sleeping person without obstructive sleep apnea, or at least sufficient to retain the position of the tongue out of the airway. It is not desirable to induce a significant contraction of many motor units, but rather to hold the base of the tongue in a normal position away from the posterior wall of the airway. In this way, the patient is not awakened by the stimulation, it is not uncomfortable, and events of obstruction of the airway are prevented entirely, rather than merely interrupted after they have occurred.

The genioglossus muscle and glossopharyngeal branches innervating it and the upper airway/pharynx are an appropriate target for the system of the invention. The motor innervation of the intrinsic muscles of the tongue is provided by the paired hypoglossal nerves (the twelfth cranial nerve). The glossopharyngeal nerve is distributed to both tongue and pharynx. It has mixed functions and supplies the posterior one-third of the tongue (at the base) and the hypoglossus muscle.

Unlike previous methods to treat obstructive sleep apnea by stimulating the genioglossus muscle or nerve branches innervating it, the methods of the invention prevent obstructive events rather than stopping obstruction after it starts.

The preceding description has been presented with references to presently preferred embodiments of the invention. Persons skilled in the art and technology to which this invention pertains will appreciate that alterations and changes in the described structures and methods can be practiced without meaningfully departing from the principle, spirit and scope of this invention.

Accordingly, the foregoing description should not be read as pertaining only to the precise structures and methods described and shown in the accompanying drawings, but rather should be read as consistent with and as support for the following claims, which are to have their fullest and fairest scope.

What is claimed is:

1. A method for preventing obstructive sleep apnea events in a patient comprising the steps of:
providing at least one electrode capable of delivering electrical stimulation;
implanting the at least one electrode in or near the patient's genioglossus muscle; and
stimulating the at least one electrode at a frequency and intensity, thereby maintaining muscle tone of the upper airway muscle and preventing sleep apnea events from occurring.

2. The method of claim 1 further comprising the step of providing an electrical pulse generator for stimulating the at least one electrode.

3. The method of claim 2 wherein the electrical pulse generator is adjustable, the method further comprising the steps of adjusting the electrical pulse generator for frequency and intensity of electrical pulses.

4. The method of claim 3 wherein the steps of adjusting the electrical pulse generator are performed remotely after the implanting step.

5. The method of claim 2 further comprising the step of starting the electrical pulse generator at a given time.

6. The method of claim 5 wherein the given time is a first given time, the method further comprising the step of stopping the electrical pulse generator at a second given time.

7. The method of claim 1 wherein the step for stimulating the at least one electrode is performed for a given time period.

8. The method of claim 7 wherein the given time period is while the patient is sleeping.

9. The method of claim 1 wherein the stimulation is a low level stimulation.

10. The method of claim 1 wherein the electrode is implanted near or in contact with a branch of the patient's glossopharyngeal nerve.

11. The method of claim 1 wherein a plurality of electrodes are implanted and stimulated.

12. The method of claim 1 wherein the at least one electrode is implanted sufficiently near the posterior one-third of the genioglossus muscle that at least some fibers of the genioglossus muscle are at least partially depolarized by the stimulation step.

13. An apparatus for preventing obstructive sleep apnea events in a patient comprising:
at least one implantable electrode adapted for implantation in or near patient's genioglossus muscle;
a stimulator adapted to deliver electrical stimuli to the electrode; and
a controller adapted to control the delivery of the electrical stimuli at a frequency and intensity, wherein the delivery of the electrical stimuli maintains muscle tone of the upper airway muscle to prevent sleep apnea events from occurring.

14. The apparatus of claim 13 wherein the controller can be adjusted to change the frequency and intensity of electrical pulses.

15. The apparatus of claim 14 wherein the controller is a programmable controller.

16. The apparatus of claim 15 further comprising a device for programming the controller from a location external from the patient.

17. The device of claim 13 comprising a plurality of electrodes.

18. The device of claim 13 further comprising a sensor for monitoring a physiological function of the patient.

19. The device of claim 18 wherein the sensor is a sensor selected from the group consisting of breathing sensors, muscle tone sensors, muscle contraction sensors, blood gas sensors, and blood pH sensors.
